(19) United States
(12) Patent Application Publication
(10) Pub. No.: US 2024/0269432 A1
(43) Pub. Date: Aug. 15, 2024

(54) Blood Draw Connector for Open Peripheral Intravenous Catheters

(71) Applicant: Becton, Dickinson and Company, Franklin Lakes, NJ (US)

(72) Inventors: Srinath B, Tamil Nadu (IN); Stephen Robert Cassidy, Mahwah, NJ (US); Balaji Kannan, Chennai (IN); Karthik MR, Bangalore (IN)

(21) Appl. No.: 18/110,098

(22) Filed: Feb. 15, 2023

(51) Int. Cl. A61M 25/00; A61B 5/15

(52) U.S. Cl. CPC A61M 25/0097; A61B 5/150992; A61M 2039/0258

(57) ABSTRACT

Provided herein is a catheter system including a catheter and a catheter hub, with the catheter hub further including a hub body having a cavity formed therein extending between a hub distal end and a hub proximal end, a hub port positioned at the hub proximal end, and a wedge positioned within the cavity adjacent the hub distal end, the wedge coupled to a proximal end of the catheter. A connector is coupled to the catheter hub and includes a proximal coupler, a distal coupler configured to mate with the hub port, and a primary lumen extending between the distal and proximal couplers that is in fluid communication with a lumen of the catheter. The distal coupler includes a collar portion configured to mate with the hub port and an extension tip extending distally out past the collar portion and into the cavity, to the wedge of the catheter hub.

### BACKGROUND OF THE INVENTION

### Field of the Invention

Provided herein are devices and systems for use in vascular access, and, in particular, a catheter connector that provides for advancement of an instrument through an open peripheral intravenous catheter.

### Description of Related Art

Vascular access devices (VADs) are used in the medical field to access peripheral vasculature of a patient for purposes of infusion therapy and/or blood withdrawal. Common types of VADs include over-the-needle peripheral intravenous catheters (PIVCs), peripherally inserted central catheters (PICCs), central venous catheters (CVCs), and midline catheters. The VAD may be indwelling for short term (days), moderate term (weeks), or long term (months to years).

Instrument delivery devices are often used with VADs to deliver an instrument into the indwelling intravenous (IV) catheter thereof, with the instrument delivery device advancing the instrument beyond the tip of the indwelling catheter. When the instrument delivery device is used to collect blood, as one example, the instrument can be in the form of a flexible tube or catheter, but the instrument may also be a guidewire, obturator, wire, electrical wiring, probe, or sensor(s), in other implementations. In some configurations, an instrument delivery device may be coupled directly to a open catheter hub (to which the indwelling catheter is secured), with the instrument delivery device operated to advance the instrument out from the instrument delivery device, through an interior of the catheter hub (i.e., through an interior cavity and a wedge that holds the catheter), and then out beyond the tip of the indwelling catheter. In other configurations, a catheter adapter or connector may serve as an interface between the catheter hub and the instrument delivery device, with a distal end of the instrument delivery device secured to the catheter adapter. When coupled to the catheter adapter, the instrument delivery device may be operated to advance the instrument out from the instrument delivery device, through a lumen of the catheter adapter, through an interior of the catheter hub, and then out beyond the tip of the indwelling catheter.

In either of the configurations described above, it is recognized that difficulties may be encountered when attempting to advance the instrument through the catheter hub. As shown in FIG. 1A, in configurations where an instrument delivery device is coupled directly to a catheter hub 1, a majority of a cavity 2 within the catheter hub 1 may remain open, and when attempting to advance an instrument 3 through this cavity 2 and into a lumen of the wedge 4 and a catheter 5, the instrument 3 may not remain aligned properly therewith. Accordingly, the instrument 3 may become trapped within the cavity 2 of the catheter hub 1 and, as the instrument 3 is further advanced, the instrument 3 may kink and double over on itself. As shown in FIG. 1B, even in configurations where a catheter connector 6 is used to connect an instrument delivery device to the catheter hub 1, a male connection portion 7 of the catheter connector 6 that couples with the catheter hub 1 may not fill a majority of a cavity 2 therein. With at least a portion of the cavity 2 remaining open, a distal end of the male connection portion 7 remains spaced apart from the wedge 4 in the catheter hub 1 and thus, when attempting to advance the instrument 3 through this cavity 2 (i.e., through the open cavity portion remaining between the male connection portion 7 and the wedge 4), the instrument 3 may again not remain aligned properly with the lumen in the wedge 4. Accordingly, the instrument 3 may become trapped within the cavity 2 of the catheter hub 1 and, as the instrument 3 is further advanced, the instrument 3 may kink and double over on itself.

Accordingly, a need exists in the art for an improved adapter or connector that provides for advancement of an instrument through the catheter connector and the catheter hub connected thereto, so as to prevent kinking of the instrument when advanced via an instrument delivery device.

### SUMMARY OF THE INVENTION

Provided herein is a catheter system that includes a catheter having a catheter distal end and a catheter proximal end and a catheter hub configured to receive a portion of the catheter therein, the catheter hub further including a hub body having a cavity formed therein extending between a hub distal end and a hub proximal end, a hub port positioned at the hub proximal end, and a wedge positioned within the cavity adjacent the hub distal end, the wedge coupled to the catheter proximal end to retain the catheter to the catheter hub. The catheter system also includes a connector coupled to the catheter hub, the connector having a connector distal end and a connector proximal end, with the connector further including a distal coupler positioned at the connector distal end and configured to mate with the hub port, a proximal coupler positioned at the connector proximal end, and a primary lumen extending between the distal coupler and the proximal coupler, the primary lumen being in fluid communication with a catheter lumen defined by the catheter when the distal coupler is coupled to the hub port. The distal coupler further includes a collar portion configured to mate with the hub port and an extension tip extending distally out past the collar portion and extending into the cavity, to the wedge of the catheter hub.

In some embodiments, a distal end of the extension tip abuts the wedge, such that a distal end of the primary lumen is directly adjacent a proximal end of the catheter lumen.

In some embodiments, the wedge includes a wedge lumen formed therein, and abutting of the distal end of the extension tip with the wedge directly aligns the primary lumen with the wedge lumen.

In some embodiments, the hub port is a female luer port and the distal coupler is a male luer connection, with the collar portion threadingly engaging the female luer port and the extension tip extending through the female luer port.

In some embodiments, the connector further includes a side port arranged in the connector between the proximal coupler and the distal coupler, the side port configured to be in fluid communication with the primary lumen.

In some embodiments, the connector is a Y-connector, with the side port extending from the connector at an angle relative to the primary lumen of between 15-165 degrees.

In some embodiments, the catheter system further includes a needle-free access connector coupled to the side port.

In some embodiments, the catheter system further includes an IV tube coupled to the side port and fluidly connected to the primary lumen, to deliver fluid to the primary lumen and onto the catheter.

In some embodiments, the proximal coupler includes a proximal access port and a split septum connector coupled to the proximal access port, the split septum connector including a first ring and a second ring spaced apart laterally from the first ring.

In some embodiments, the catheter system further includes a blood draw device coupled to the proximal coupler of the connector.

In some embodiments, the blood draw device includes a secondary catheter, an introducer having a proximal end and a distal end, and a top surface and a bottom surface, that define an inner volume configured to movably receive the secondary catheter, an actuator movably coupled to the introducer to move relative to the introducer to move a distal end of the secondary catheter from a position within the introducer to a position outside the introducer housing and past the catheter distal end of the catheter, and a lock positioned at the distal end of the introducer that is coupled to the proximal coupler of the connector.

In some embodiments, the lock includes a pair of locking arms, with the pair of locking arms engaging the split septum connector by snapping-in between the first ring and the second ring.

In some embodiments, the primary lumen has a linear shape, such that the secondary catheter may be advanced through the primary lumen in a straight line when the blood draw device is coupled to the proximal coupler of the connector.

In some embodiments, wherein extension of the extension tip distally to the wedge of the catheter hub provides concentricity between the secondary catheter and the catheter.

In some embodiments, the catheter hub is an open peripheral intravenous catheter (PIVC) catheter hub.

Also provided herein is a catheter system that includes a catheter having a catheter distal end and a catheter proximal end and a catheter hub configured to receive a portion of the catheter therein, the catheter hub further including a hub body having a cavity formed therein extending between a hub distal end and a hub proximal end, a hub port positioned at the hub proximal end provided as a female luer connection, and a wedge positioned within the cavity adjacent the hub distal end, the wedge coupled to the catheter proximal end to retain the catheter to the catheter hub. The catheter system also includes a connector coupled to the catheter hub, the connector having a connector distal end and a connector proximal end, with the connector further including a male luer connection at the connector distal end configured to mate with the female luer connection. The male luer connection includes an extension tip that extends into the cavity and substantially therethrough, to abut the wedge.

In some embodiments, the male luer connection includes a collar positioned about a portion of the extension tip, the collar having a threaded inner surface configured to engage a threaded outer surface of the female luer connection.

In some embodiments, the connector further includes a proximal coupler positioned at the connector proximal end, with the proximal coupler having a proximal access port and a split septum connector coupled to the proximal access port, the split septum connector including a first ring and a second ring spaced apart laterally from the first ring.

In some embodiments, the catheter system further includes a blood draw device coupled to the proximal coupler of the connector, with the blood draw device including a secondary catheter, an introducer having a proximal end and a distal end, and a top surface and a bottom surface, that define an inner volume configured to movably receive the secondary catheter, an actuator movably coupled to the introducer to move relative to the introducer to move a distal end of the secondary catheter from a position within the introducer to a position outside the introducer housing and past the catheter distal end of the catheter, and a lock positioned at the distal end of the introducer that is coupled to the proximal coupler of the connector.

In some embodiments, the lock includes a pair of locking arms, with the pair of locking arms engaging the split septum connector by snapping-in between the first ring and the second ring.

### BRIEF DESCRIPTION OF THE DRAWINGS

FIG. 1A is a cross-sectional view of a catheter hub, with an instrument being advanced therethrough, as known in the prior art;

FIG. 1B is a cross-sectional view of a catheter hub and associated catheter connector, with an instrument being advanced therethrough, as known in the prior art;

FIG. 2 is a perspective view of a catheter system, according to an aspect or embodiment of the present application;

FIG. 3 is a top view of a portion of the catheter system of FIG. 2;

FIG. 4 is a cross-sectional view of the catheter system of FIG. 2;

FIG. 5 is a cross-sectional view of the catheter system of FIG. 2, showing an instrument in a first position; and

FIG. 6 is a cross-sectional view of the catheter system of FIG. 2, showing an instrument in a second position.

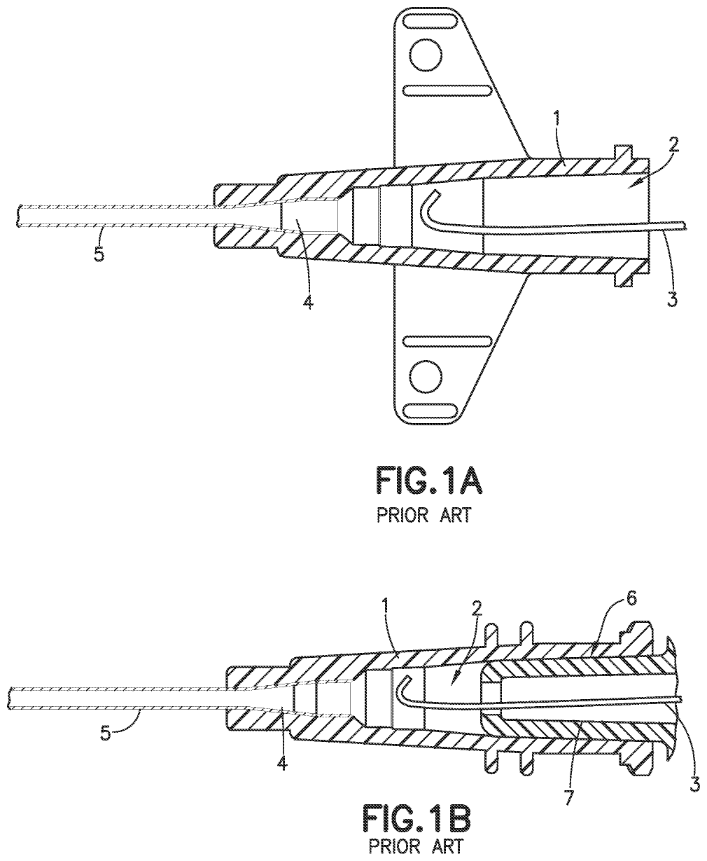

### DESCRIPTION OF THE INVENTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a catheter insertion device in preparation for or during use. Similarly, in this application, the terms “in the distal direction” and “distally” mean in the direction toward the distal tip of the needle or catheter of the system, and the terms “in the proximal direction” and “proximally” mean in the direction opposite the direction of the distal tip of the needle or catheter.

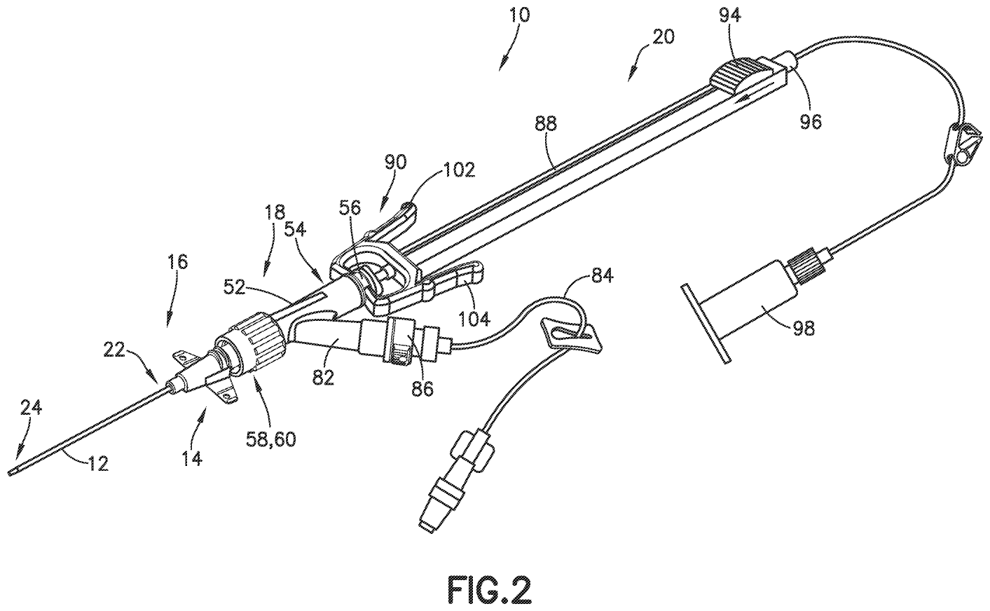

Referring to FIG. 2, a catheter system 10 is shown, according to an aspect or embodiment of the disclosure. The catheter system 10 includes a catheter 12 and associated catheter hub 14 that may collectively provide an open PIVC catheter 16. The catheter system 10 further includes a catheter adapter or connector 18 (hereinafter “connector 18”) to which an instrument delivery device 20 and/or additional extension set or fluid delivery system may be connected, to provide for delivery of an instrument and/or fluid into and through the catheter 12.

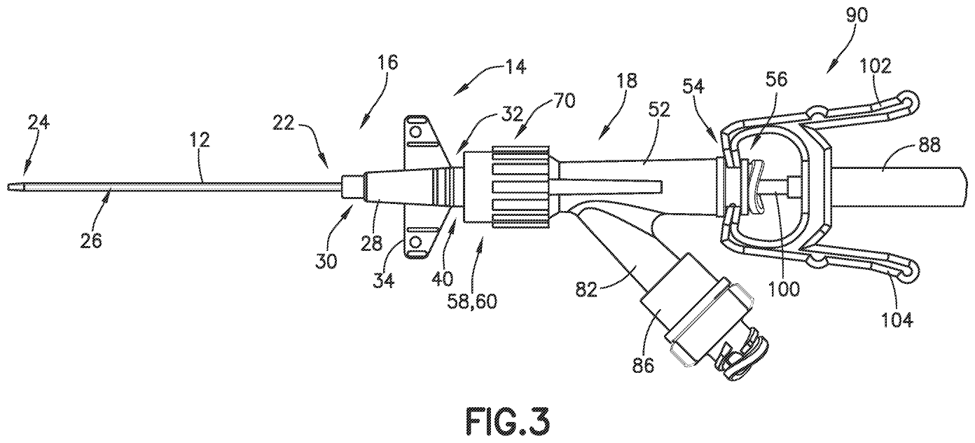

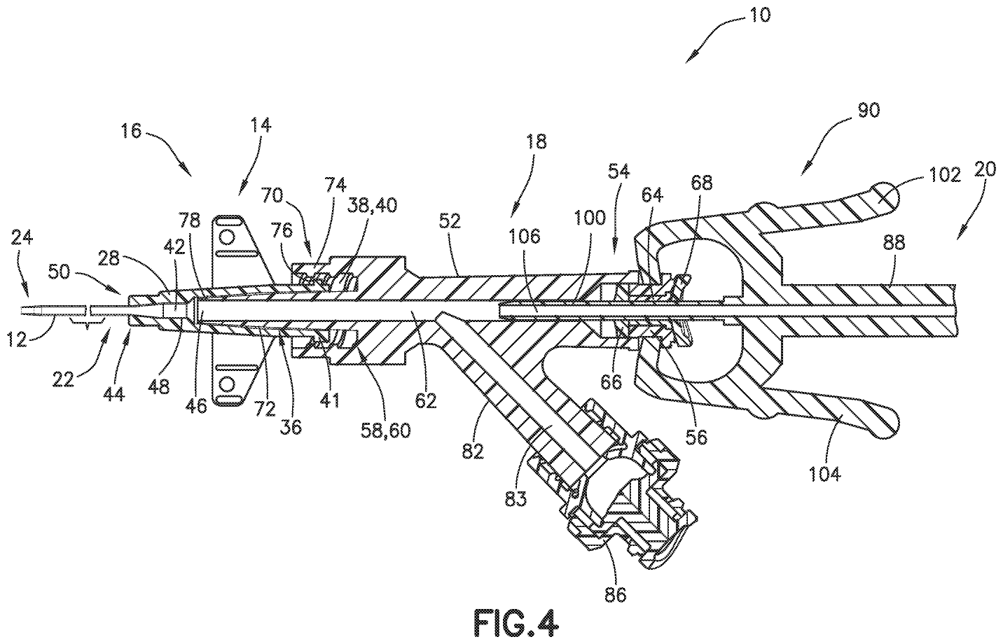

As shown in FIG. 2 and also in FIGS. 3 and 4, the open PIVC catheter 16 includes a catheter 12 and catheter hub 14 that provide intravenous access to the vasculature of a patient. In some embodiments, the catheter 12 may include a peripheral intravenous catheter, but it is recognized that catheter 12 could also be provided as a midline catheter, or a peripherally-inserted central catheter. Catheter 12 may be formed of any suitable material and may be of any useful length, as known to those of skill in the art. A proximal end 22 of the catheter 12 is coupled to catheter hub 14 and extends distally away therefrom to a distal end 24 of the catheter 12. The catheter 12 defines a lumen 26 therein via which fluids may be administered to a patient, an instrument may be introduced into the vasculature of the patient, or blood may be drawn from the patient.

The catheter hub 14 is constructed of a main hub body 28 having a distal end 30 and a proximal end 32, along with stabilizing wings 34 extending out from the main hub body 28 that provide for securing of the hub to the skin of a patient. The main hub body 28 defines a cavity 36 therein that extends between the distal end 30 and the proximal end 32 of the catheter hub 14. According to some aspects or embodiments, the cavity 36 is configured as a tapered cavity 36 and forms part of a hub port 38 included at the proximal end of the catheter hub 14—with the hub port 38 configured as a female luer connection 40. The hub port 38, in addition to including the tapered cavity 36, also includes threads 41 formed on an outer surface of the main hub body 28 that are provided adjacent the proximal end 32 of the catheter hub 14. The tapered cavity 36 and threads 41 of hub port 38 provide for coupling of a male luer connection to the catheter hub 14, with the male luer connection coupling with the female luer connection 40 of hub port 38, as will be explained in further detail below.

As shown in FIG. 4, catheter hub 14 further includes a wedge 42 positioned within the cavity 36 of main hub body 28. The wedge 42 is positioned within the cavity 36 adjacent distal end 30 of the catheter hub 14 and includes an outer surface contoured to match contours of the cavity 36, such that the wedge 42 may be seated within the cavity 36. The wedge 42 includes openings 44, 46 formed therein at opposing distal and proximal ends thereof, with a wedge lumen 48 extending between the distal and proximal openings 44, 46. The wedge 42 is positioned within cavity 36 so as to be aligned with a distal opening 50 in the main hub body 28, with the distal opening 44 in wedge 42 aligned with the distal opening 50 in the main hub body 28. With the openings of the wedge 42 and main hub body 28 aligned, the catheter 12 extends through the distal opening 50 in the main hub body 28 and into the distal opening 44 in wedge 42—with the distal opening 44 in wedge 42 sized such that the catheter 12 is secured within the opening 44 and secured to the wedge 42, thereby anchoring the catheter 12 to the catheter hub 14.

The connector 18 of catheter system 10 is connected to the proximal end 32 of catheter hub 14 and serves as an adaptor or connection between the catheter hub 14 and the instrument delivery device 20. The connector 18 is formed of a main connector body 52 that includes a proximal port 54 with a proximal coupler 56, a distal port 58 with a distal coupler 60, and defines a primary lumen 62 extending through or otherwise in fluid communication with the couplers 56 and 58. In accordance with aspects of the disclosure, the primary lumen 62 has a linear shape and extends straight through the main connector body 52, so as to facilitate advancement of an instrument through the connector 18, as will be explained in greater detail below.

In accordance with some aspects and embodiments, the proximal port 54 is configured as a receptacle that receives a proximal coupler 56 therein. The proximal coupler 56 may be configured as a split-septum, needle-free connector (NFC) or the like that is fitted partially within the proximal port 54 and includes a split septum 64 therein that serves as an access control feature whereby a peripheral device (e.g., a blood draw device or a vascular access probe for in-vein digital measurement of patient data such as temperature, pH, lactate, and/or other blood-based measurements) can be physically and fluidically coupled to the connector 18, with the split-septum connector allowing the connector 18 to be accessed multiple times by the peripheral device. In some embodiments, where the peripheral device is a blood draw device that introduces a secondary catheter into catheter 12, the split-septum connector 56 functions as a blood control feature that may be accessed multiple times by the blood draw device to draw the blood from a vein of the patient. The split-septum connector 56 also includes a pair of connection rings 66, 68 formed about a perimeter thereof, with a first ring 66 and a second ring 68 spaced laterally apart. The connection rings 66, 68 provide for engagement of the instrument delivery device 20 to the connector 18, as will be explained in further detail below.

In accordance with some aspects and embodiments, the distal port 58 of connector 18 provides a distal coupler 60 that may be at least partially formed integrally with the main connector body 52, with the distal coupler 60 provided as a male luer connection 70. The male luer connection 70 generally includes an extension tip 72 and an annular shield or collar portion 74, with the collar portion 74 positioned around a portion of the extension tip 72 (i.e., extension tip 72 extends out past collar portion 74). The collar portion 74 may be configured to engage the female luer connection 40 of the catheter hub 14 via a twist-turn or threading engagement, with one or more threads 76 formed on an interior surface of the collar portion 74 mating with threads 41 on the female luer connection 40 of catheter hub 14. As shown in the illustrated embodiment, the collar portion 74 may be configured as a “fixed” collar that is integrally formed with the main connector body 52 and with respect to the extension tip 72. However, it is recognized that in other embodiments, the collar portion 74 may be configured as a “spinning” collar that is formed separate from the connector body 52 and extension tip 72, such that the collar portion 74 is allowed to rotate relative thereto, so as to enable rotation of the collar portion 74 when threadingly engaging the collar portion 74 with the catheter hub 14.

As shown in FIG. 4, the extension tip 72 is formed as an elongate member that extends out past collar portion 74 and into the cavity 36 of catheter hub 14. The extension tip 72 may be configured to have a tapered outer surface received by the corresponding tapered cavity 36 of the female luer connection 40 of catheter hub 14. A length of the extension tip 72 is such that a distal end 78 of the extension tip 72 extends entirely through the cavity 36 and into contact with the wedge 42 of catheter hub 14—i.e., the extension tip 72 abuts the wedge 42. With the extension tip 72 abutting the wedge 42 in this manner, a distal opening 80 of the primary lumen 62—which runs through the extension tip 72—is brought directly adjacent the wedge lumen 48 and into proximity with the proximal end 22 of the catheter 12. With the extension tip 72 inter-fitted with the cavity 36 of catheter hub 14 in this manner, the primary lumen 62 in extension tip 72 (and more generally in connector 18) is directly aligned with the wedge lumen 48. Accordingly, the distal coupler 60 can be physically and fluidically coupled to catheter hub 14 such that the primary lumen 62 of the connector 18 is at least selectively in fluid communication with the catheter 12 through the catheter hub 14.

The connector 18 also includes and/or defines one or more additional ports, such as a side port 82. The side port 82 may be formed in a desired position along a length of the main connector body 52, between the distal port 58 and the proximal port 54, to allow for a reduced length of the connector 18 and/or to facilitate flushing and/or fluid transfer via the side port 82. The side port 82 defines a lumen 83 that is in fluid communication with the primary lumen 62 between the distal coupler 60 and proximal coupler 56. As such, side port 82 can provide access to the primary lumen 62 between distal coupler 60 and proximal coupler 56, which in turn can provide access to a device (e.g., a catheter hub 14) that is coupled to the distal coupler 60.

In some embodiments, the arrangement of side port 82 can be such that the connector 18 forms, for example, a Y-connector or a T-connector. More particularly, side port 82 can be disposed substantially perpendicular (e.g., about 90 degrees) to the lumen of the connector 18 and near or adjacent the proximal coupler 56. In non-limiting embodiments, side port 82 extends from connector 18 at an angle, relative to the primary lumen 62 between distal coupler 60 and proximal coupler 56, that is not 90 degrees (e.g., side port 82 extends at an angle of, for example and without limitation, 15-165 degrees, with all values and subranges therebetween inclusive). In non-limiting embodiments, more than one side port 82 is included in connector 18.

As shown in FIG. 2, in some embodiments, extension tubing 84 may be connected to side port 82 such that the extension tubing 84 is in fluid communication with the lumen of the side port 82. The side port 82 and/or the extension tubing 84 can be and/or can form at least a portion of a fluid line that can be used to deliver fluid, remove fluid, flush fluid, and/or the like, such as receiving fluid from an attached IV bag (not shown), as one example. In such embodiments, an arrangement in which side port 82 is disposed adjacent to the proximal coupler 56 can enable flushing of the proximal coupler 56, any valve(s) included therein, and/or a space between any valve(s) and an inner surface of the connector 18 (e.g., defining at least a portion of the primary lumen 62).

In other embodiments, and as shown in FIGS. 2-4, a NFC 86 may be coupled to the side port 82 to provide controlled access to the connector 18, with extension tubing 84 connected to the side port 82 through NFC 86. The NFC 86 may be configured as a split-septum NFC such as, e.g., Q-Syte™ or SmartSite™ NFCs from Becton, Dickinson and Co., or any other appropriate split-septum NFC.

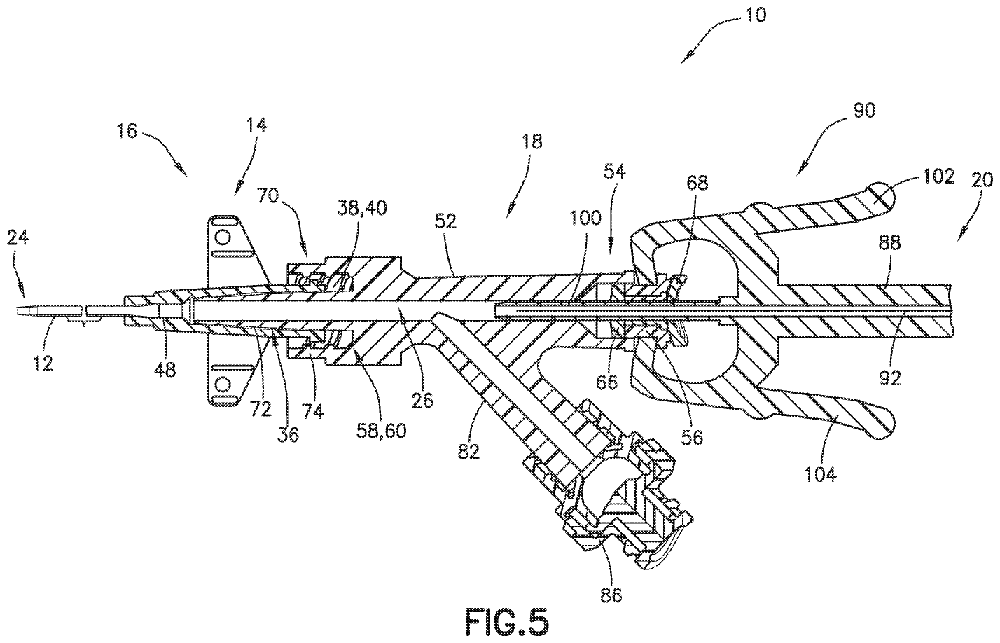

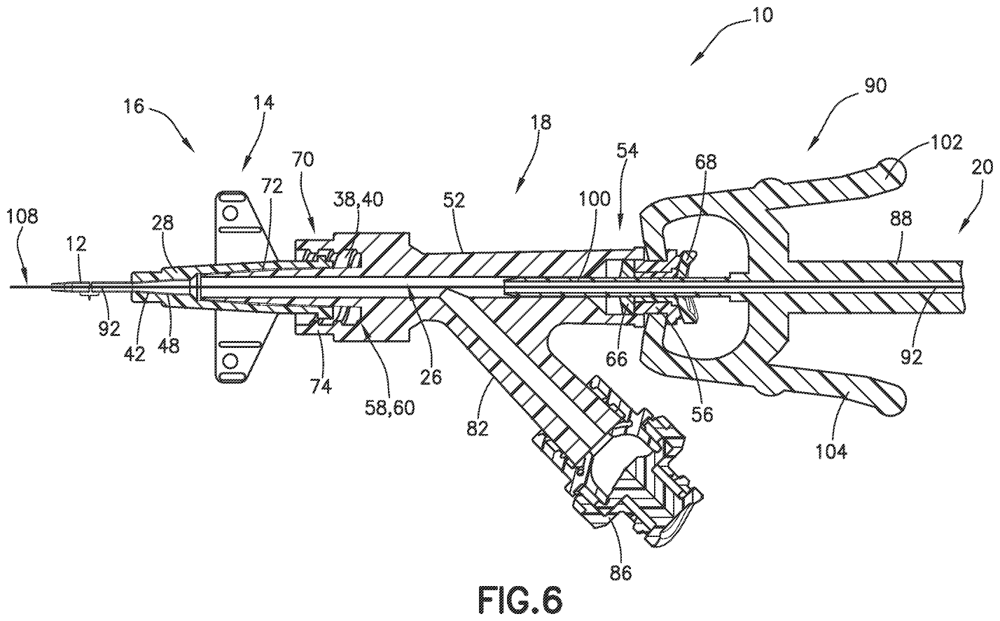

As shown in FIG. 2 and in FIGS. 5 and 6, the instrument delivery device 20 of catheter system 10 is coupled to the proximal coupler 56 of connector 18. The instrument delivery device 20 includes an introducer 88, a lock 90, an instrument 92 (FIGS. 5 and 6), and an actuator 94. The instrument 92 may be any of a number of devices suitable for insertion into the vasculature of the patient, including a secondary catheter, guidewire, or probe, as examples. A portion of the actuator 94 is configured to be advanced along a top surface of the introducer 88, which in turn, facilitates the advancing of the instrument 92 through the introducer 88, the lock 90, the connector 18, and distally out through and past the catheter 12. In some embodiments, a luer adapter, luer lock access device (LLAD), or other connecting device 96 may be provided at a proximal end of the introducer 88 to enable connection of another component 98 to the instrument delivery device 20, such as a syringe or vacutainer to facilitate a blood draw. According to one embodiment, the instrument delivery device 20 acts as a blood draw device that inserts a secondary catheter through the catheter 12 to facilitate a blood draw from the patient, with blood collected in the syringe or vacutainer attached to the device, and thus reference hereafter is made to a blood draw device 20 and secondary catheter 92.

As shown in more detail in FIGS. 3 and 4, the lock 90 of the blood draw device 20 is configured to be physically and fluidically coupled to the introducer 88 and configured to couple the introducer 88 to the connector 18. The lock 90 has a blunt cannula 100, a first arm 102, and a second arm 104, with the lock 90 also defining a lumen 106 extending through the blunt cannula 100. The blunt cannula 100 is disposed between the first arm 102 and the second arm 104 and has a length that is sufficient to extend through the proximal connector 56 (i.e., split-septum connector) of proximal port 54 and at least partially into the primary lumen 62 of connector 18. Furthermore, the blunt cannula 100 can have an inner diameter (a diameter of a surface at least partially defining the lumen 106) that is similar to or slightly larger than an outer diameter of a portion of the secondary catheter 92. Thus, the lumen 106 of the lock 90 can receive a portion of the secondary catheter 92 when the secondary catheter is advanced therethrough.

As described above, at least a portion of the secondary catheter 92 is movably disposed within the introducer 88. A proximal end portion of the secondary catheter 92 is coupled to the actuator 94 and, in this manner, the actuator 94 can be moved relative to the introducer 88 to move the secondary catheter 92 between a first position, in which the secondary catheter 92 is disposed within the introducer 88 (e.g., the entire secondary catheter 92 is disposed within the introducer 88 or within the introducer 88 and the lock 90) and a second position, in which the distal end portion 108 of the secondary catheter 92 is at least partially disposed in a position distal to the lock 90 and the connector 18 when the lock 90 is coupled to the connector 18.

Referring now to FIGS. 5 and 6, changing of the blood draw device 20 between a first configuration and second configuration is shown. The blood draw device 20 can be in the first configuration prior to use and can be transitioned by a user (e.g., a doctor, physician, nurse, technician, phlebotomist, and/or the like) from the first configuration (FIG. 5) to the second configuration (FIG. 6) to dispose at least the distal end portion 108 of the secondary catheter 92 in a distal position relative to the introducer 88 (e.g., within or distal to the indwelling catheter 12).

The blood draw device 20 is shown in the first configuration of FIG. 5 when the secondary catheter 92 is disposed in the first position within the introducer 88. In some embodiments, substantially the entire secondary catheter 92 is disposed within the introducer 88 when the secondary catheter 92 is in the first position. In other embodiments, the secondary catheter 92 is disposed within the introducer 88 and the lock 90 when secondary catheter 92 is in the first position.

The actuator 94 (FIG. 2) is disposed in a proximal position when the blood draw device 20 is in the first configuration, and the user may engage the actuator 94 to it relative to the introducer 88, which in turn, moves the secondary catheter 92 from the first position (e.g., disposed within the introducer 88) toward the second position. In this manner, the secondary catheter 92 is moved through the inner volume of the introducer 88 and through the lumen 106 of the lock 90.

The blood draw device 20 is in the second configuration of FIG. 6 when the secondary catheter 92 is disposed in the second position. The second position of the secondary catheter 92 is reached when the distal end portion 108 of the secondary catheter 92 is placed in a desired position relative to a distal end of the connector 18 and into/through the catheter 12. In some instances, for example, a distal end 108 of the secondary catheter 92 can be substantially flush with a distal end 24 of the catheter 12 when the secondary catheter 92 is in the second position. In other instances, the distal end 108 of the secondary catheter 92 can extend a predetermined distance beyond the distal end 24 of the catheter 12 (e.g., distal to the distal end 24 of the catheter 12), such that the distal end 108 of the secondary catheter 92 is positioned within the vein at a predetermined distance beyond the distal end 24 of the catheter 12 (e.g., a position within a vein that is substantially free from debris (e.g., fibrin/blood clots) otherwise surrounding the distal end 24 of the catheter 12).

With the secondary catheter 92 in the second position (e.g., with the blood draw device 20 in the second configuration shown, for example, in FIG. 6), the user can establish fluid communication between a component 98 (e.g., fluid reservoir, fluid source, syringe, and/or the like) and the secondary catheter 92. With the secondary catheter 92 in fluid communication with the component 98, the blood draw device 20 can then aspirate a volume of blood from the vein based, at least in part, on disposing the distal end 108 of the secondary catheter 92 at the predetermined and/or desired distance beyond the distal end 24 of the indwelling catheter 12.

Beneficially, the construction of connector 18 and the mating thereof with catheter hub 14 facilitates advancement of the secondary catheter 92 from the first position to the second position. That is, with the extension tip 72 having a length such that it extends entirely through the cavity 36 of catheter hub 14 and into contact with the wedge 42, i.e., the extension tip 72 abuts the wedge 42, a distal opening 80 of the primary lumen 62 (that runs through the extension tip 72) is brought directly adjacent to and into alignment with the wedge lumen 48. Accordingly, the secondary catheter 92 can be advanced through the primary lumen 62 of connector 18 and through the wedge lumen 48 of wedge 42 in a straight line—without any opportunity for the secondary catheter 92 to deflect or otherwise become trapped/caught within the cavity 36 of the catheter hub 14. The structure of connector 18 and mating thereof with catheter hub 14 thereby maintains concentricity between the secondary catheter 92 and the catheter 12, therefore assisting with advancement of the secondary catheter 92.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A catheter system comprising:
a catheter having a catheter distal end and a catheter proximal end;
a catheter hub configured to receive a portion of the catheter therein, the catheter hub comprising:
a hub body having a hub distal end and a hub proximal end, the hub body having a cavity formed therein extending between the hub distal end and the hub proximal end;
a hub port positioned at the hub proximal end; and
a wedge positioned within the cavity adjacent the hub distal end, the wedge coupled to the catheter proximal end to retain the catheter to the catheter hub; and
a connector coupled to the catheter hub, the connector having a connector distal end and a connector proximal end, with the connector comprising:
a distal coupler positioned at the connector distal end and configured to mate with the hub port;
a proximal coupler positioned at the connector proximal end; and
a primary lumen extending between the distal coupler and the proximal coupler, the primary lumen being in fluid communication with a catheter lumen defined by the catheter when the distal coupler is coupled to the hub port;
wherein the distal coupler comprises:
a collar portion configured to mate with the hub port; and
an extension tip extending distally out past the collar portion and extending into the cavity, to the wedge of the catheter hub.

2. The catheter system of claim 1, wherein a distal end of the extension tip abuts the wedge, such that a distal end of the primary lumen is directly adjacent a proximal end of the catheter lumen.

3. The catheter system of claim 2, wherein the wedge includes a wedge lumen formed therein, and abutting of the distal end of the extension tip with the wedge directly aligns the primary lumen with the wedge lumen.

4. The catheter system of claim 1, wherein the hub port comprises a female luer port and the distal coupler comprises a male luer connection, with the collar portion threadingly engaging the female luer port and the extension tip extending through the female luer port.

5. The catheter system of claim 1, wherein the connector further comprises a side port arranged in the connector between the proximal coupler and the distal coupler, the side port configured to be in fluid communication with the primary lumen.

6. The catheter system of claim 5, wherein the connector comprises a Y-connector, with the side port extending from the connector at an angle relative to the primary lumen of between 15-165 degrees.

7. The catheter system of claim 5, further comprising a needle-free access connector coupled to the side port.

8. The catheter system of claim 5, further comprising an IV tube coupled to the side port and fluidly connected to the primary lumen, to deliver fluid to the primary lumen and onto the catheter.

9. The catheter system of claim 1, wherein the proximal coupler comprises:
a proximal access port; and
a split septum connector coupled to the proximal access port, the split septum connector comprising a first ring and a second ring spaced apart laterally from the first ring.

10. The catheter system of any of claim 9, wherein the catheter system further comprises a blood draw device, the blood draw device coupled to the proximal coupler of the connector.

11. The catheter system of claim 10, wherein the blood draw device comprises:
a secondary catheter;
an introducer having a proximal end and a distal end, and a top surface and a bottom surface, that define an inner volume configured to movably receive the secondary catheter;
an actuator movably coupled to the introducer, the actuator configured to move relative to the introducer to move a distal end of the secondary catheter from a position within the introducer to a position outside the introducer housing and past the catheter distal end of the catheter; and
a lock positioned at the distal end of the introducer that is coupled to the proximal coupler of the connector.

12. The catheter system of claim 11, wherein the lock comprises a pair of locking arms, and wherein the pair of locking arms engage the split septum connector by snapping-in between the first ring and the second ring.

13. The catheter system of claim 12, wherein the primary lumen has a linear shape, such that the secondary catheter may be advanced through the primary lumen in a straight line when the blood draw device is coupled to the proximal coupler of the connector.

14. The catheter system of claim 11, wherein extension of the extension tip distally to the wedge of the catheter hub provides concentricity between the secondary catheter and the catheter.

15. The catheter system of claim 1, wherein the catheter hub comprises an open peripheral intravenous catheter (PIVC) catheter hub.

16. A catheter system comprising:
a catheter having a catheter distal end and a catheter proximal end;
a catheter hub configured to receive a portion of the catheter therein, the catheter hub comprising:
a hub body having a hub distal end and a hub proximal end, the hub body having a cavity formed therein extending between the hub distal end and the hub proximal end;
a hub port positioned at the hub proximal end, the hub port comprising a female luer connection; and
a wedge positioned within the cavity adjacent the hub distal end, the wedge coupled to the catheter proximal end to retain the catheter to the catheter hub; and
a connector coupled to the catheter hub, the connector having a connector distal end and a connector proximal end, the connector comprising a male luer connection at the connector distal end configured to mate with the female luer connection;
wherein the male luer connection comprises an extension tip that extends into the cavity and substantially therethrough, to abut the wedge.

17. The catheter system of claim 16, wherein the male luer connection comprises a collar positioned about a portion of the extension tip, the collar having a threaded inner surface configured to engage a threaded outer surface of the female luer connection.

18. The catheter system of claim 16, wherein the connector further comprises a proximal coupler positioned at the connector proximal end, the proximal coupler comprising:
a proximal access port; and
a split septum connector coupled to the proximal access port, the split septum connector comprising a first ring and a second ring spaced apart laterally from the first ring.

19. The catheter system of claim 18, wherein the catheter system further comprises a blood draw device coupleable to the proximal coupler of the connector, the blood draw device comprising:
a secondary catheter;
an introducer having a proximal end and a distal end, and a top surface and a bottom surface, that define an inner volume configured to movably receive the secondary catheter;
an actuator movably coupled to the introducer, the actuator configured to move relative to the introducer to move a distal end of the secondary catheter from a position within the introducer to a position outside the introducer housing and past the catheter distal end of the catheter; and
a lock positioned at the distal end of the introducer that is coupled to the proximal coupler of the connector.

20. The catheter system of claim 19, wherein the lock comprises a pair of locking arms, and wherein the pair of locking arms engage the split septum connector by snapping-in between the first ring and the second ring.
